(19) United States
(12) Patent Application Publication
(10) Pub. No.: US 2026/0360494 A1
(43) Pub. Date: Nov. 12, 2026

(54) SYSTEM AND METHOD FOR PREDICTING THE RESOLUTION OF METABOLIC DYSFUNCTION-ASSOCIATED STEATOHEPATITIS AND THE IMPROVEMENT OF LIVER FIBROSIS

(71) Applicant: GENFIT, LOOS (FR)

(72) Inventors: YACINE HAJJI, LILLE (FR); JÉRÉMY MAGNANENSI, LILLE (FR); ZOUHER MAJD, ENNETIERES-EN-WEPPES (FR)

(21) Appl. No.: 19/200,012

(22) Filed: May 6, 2025

(51) Int. Cl. G16H 20/10; G16H 10/60; G16H 50/70

(52) U.S. Cl. CPC G16H 20/10; G16H 10/60; G16H 50/70

(57) ABSTRACT

The present invention relates to a system and a computer program for evaluating the efficacy of a treatment of metabolic dysfunction-associated steatohepatitis (MASH) and of liver fibrosis in a subject with MASH, and/or for predicting the resolution of MASH and the improvement of liver fibrosis in a subject with MASH.

The present invention relates to a system and a computer program for evaluating the efficacy of a treatment of metabolic dysfunction-associated steatohepatitis (MASH) and of liver fibrosis in a subject with MASH, and/or for predicting the resolution of MASH and the improvement of liver fibrosis in a subject with MASH.

### BACKGROUND

Initiated by presence of steatosis in the liver, metabolic dysfunction-associated steatotic liver disease (MASLD), formerly known as non-alcoholic fatty liver disease (NAFLD), is the liver disease with most cases in the world (~25%) (Cotter TG and Rinella M. Gastroenterology. 2020 May; 158 (7): 1851-1864). It can progress to MASH, formerly named nonalcoholic steatohepatitis or NASH. MASH is the worst form of MASLD. MASH is a silent disease which can progress to cirrhosis and liver-related events. At-risk MASH corresponds to patients with a fibrosis stage of 2 or more, and a MASLD-Activity-Score (MAS) of 4 points or more, with at least 1 point in each MAS components (steatosis, lobular inflammation, ballooning). This population of at-risk MASH patients has an increased risk of disease progression, associated with liver-related events and mortality.

Both U.S. Food and Drug Administration (FDA) and European Medicines Agency (EMA) defined the endpoints of MASH clinical trials as the resolution of MASH and/or the improvement of liver fibrosis. To date, resmetirom has already been approved for the treatment of patients with MASH and fibrosis F2-3. Numerous drug trials are also ongoing in this population.

The method NIS2+® described in WO2023/194593 is a validated blood-based biomarker panel to identify at-risk MASH (MAS>4; F>2) with robust ruling-in/out performances. However, in the context of the treatment of MASH, it is important to be able to predict early enough whether a MASH patient has achieved MASH resolution and/or liver fibrosis improvement to improve handling of patients in standard of care. In addition, numerous ongoing MASH clinical trials raise the necessity to easily and efficiently evaluate the efficacy of treatments in these clinical trials.

There is therefore an increased need for a simple and reliable non-invasive tool for evaluating the efficacy of a treatment of MASH in a subject with MASH, and/or for predicting the resolution of MASH in a subject with MASH, which is one of the two endpoints for MASH clinical trials.

### BRIEF DESCRIPTION OF THE FIGURES

FIG. 1 is a schematic view of the invention.

FIG. 2 is another schematic view of the invention.

### DESCRIPTION OF THE INVENTION

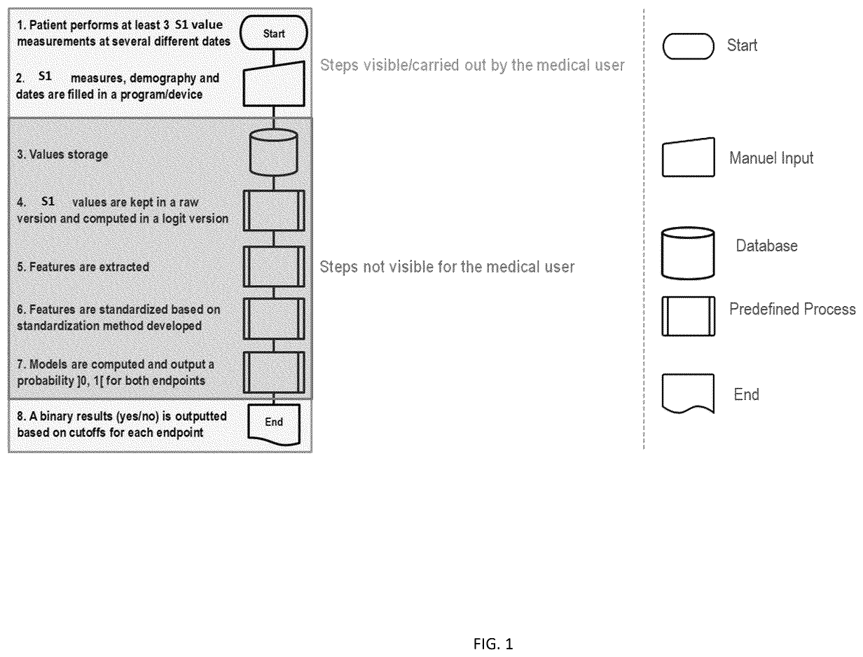

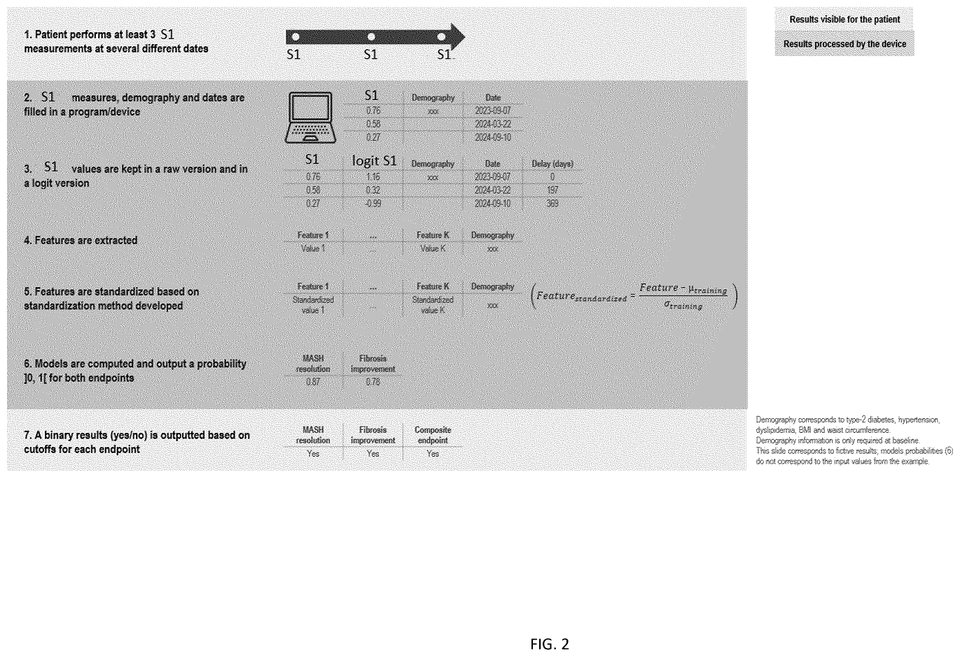

The present invention is as represented in FIGS. 1 and 2.

In one aspect, the invention relates to computer program comprising instructions that, when executed by a central processing unit (CPU), causes the central processing unit to evaluate the efficacy of a treatment of metabolic dysfunction-associated steatohepatitis (MASH) and/or to predict the resolution of MASH and the improvement of liver fibrosis in a subject with MASH.

As used herein, the term “predict” refers to the determination of the evolution of an existing disease or medical condition in a subject without performing an invasive diagnostic method, e.g. a biopsy.

According to the invention, the computer program causes the CPU to:
a) stock or calculate S1 values obtained at three or more timepoints of a subject with MASH;
b) calculate the corresponding S1 value logits;
c) stock the information relating to Type-2 diabetes comorbidity at T₀, dyslipidemia comorbidity at T₀, waist circumference of the subject at T₀ and BMI index at T₀;
d) calculate the values of 27 features X1-X27;
e) combine the values of at least 19 features among the features X1-X23 in a mathematical function to calculate a S2 score for the subject;
f) combine the values at least 6 features among the features X2, X5, X24, X25, X26, X17, X22 and X27 in a mathematical function to calculate a S3 score for the subject;
g) compare said S2 score with a reference S2 value and said S3 score with a reference S3 value, wherein:
   the subject's S2 score higher than the reference S2 value and the subject's S3 score higher than the reference S3 value indicate that the subject achieved MASH resolution and liver fibrosis improvement and/or the treatment of MASH and liver fibrosis in the subject is efficient;
   the subject's S2 score lower than the reference S2 value and the subject's S3 score higher than the reference S3 value indicate that the subject achieved liver fibrosis improvements and that the subject does not achieve MASH resolution;
   the subject's S2 score higher than the reference S2 value and the subject's S3 score lower than the reference S3 indicate that the subject achieved MASH resolution and that the subject does not achieve liver fibrosis improvement;
   the subject's S2 score lower than the reference S2 value and the subject's S3 score lower than the reference S3 score value indicate that the subject achieved neither MASH resolution nor liver fibrosis improvement and/or the treatment of MASH and liver fibrosis in the subject is not efficient;
wherein the features X1-X27 are:
   X1: goodness of exponential fit to embedding distance distribution of S1 logit values,
   X2: time reversibility of S1 logit values,
   X3: autocorrelation of differentiated time-series of S1 logit values,
   X4: 5-bin histogram mode of S1 logit values,
   X5: Proportion of S1 logit values inside a given circular boundary in a 2-d embedding space,
   X6: 10-bin histogram mode of S1 logit values,
   X7: Ljung-box test statistic of S1 logit values,
   X8: squared coefficient of variation of non-zero observations of S1 logit values,
   X9: shanon entropy of S1 logit values,
   X10: strength of trend of S1 logit values,
   X11: S1 scores percentage change between the last timepoint T_{End} and the first timepoint T₀,
   X12: whether the S1 score at T_{End} is <0.46, value 1 being assigned if the answer is yes, 0 being assigned if the answer is no,
   X13: whether the S1 scores of the subject reduce from ≥0.68 at T₀ to <0.46 at T_{End}, value 1 being assigned if the answer is yes, 0 being assigned if the answer is no,
   X14: whether the S1 score of the subject progresses from <0.46 at T₀ to ≥0.68 at T_{End}, value 1 being assigned if the answer is yes, 0 being assigned if the answer is no,
   X15: whether S1 stage is increased between T₀ and T_{End}, value 1 being assigned if the answer is yes, 0 being assigned if the answer is no,
   X16: S1 logit at T_{End},
   X17: mean of the values of S1 logit at all timepoints,
   X18: mean of all S1 logit scores delta between every two neighboring points,
   X19: standard deviation of S1 logit values at each timepoint,
   X20: whether the gender of the subject is male, value 1 being assigned if the answer is yes, 0 being assigned if the answer is no,
   X21: the body mass index (BMI) of the subject at T₀,
   X22: whether the subject has Type-2 diabetes comorbidity at T₀, value 1 being assigned if the answer is yes, 0 being assigned if the answer is no,
   X23: whether the subject has dyslipidemia comorbidity, value 1 is assigned if the answer is yes, 0 being assigned if the answer is no,
   X24: Centroid frequency of S1 logit values,
   X25: whether the S1 score at the first timepoint T₀ is <0.46, value 1 being assigned if the answer is yes, 0 being assigned if the answer is no,
   X26: whether S1 stage decreases between T₀ and T_{End}, value 1 being assigned if the answer is yes, 0 being assigned if the answer is no,
   X27: waist circumference of the subject at T₀,
wherein the score S1 is obtained from a logistic function:

$$S 1 = \frac{1}{1 + exp ( - Y )}$$

wherein

Y=B0+B1*log 10(miR-34a-5p(Fold))+B2*log 10(YKL-40 (ng/ml))+B3*Gender+B4*log 10(miR-34a-5p(Fold))*Gender
wherein
Gender is 0 if the subject is a female, or 1 if the subject is a male;
B0 is comprised between −3 and 3, in particular between −2 and 2;
B1 is comprised between 1 and 5, in particular between 2 and 4;
B2 is comprised between 0 and 4.5, in particular between 0.5 and 3;
B3 is comprised between −2 and 2, in particular between −1 and 1; and
B4 is comprised between −1 and 2, in particular between 0 and 2.

Within the context of the present invention, a “subject with MASH” or a “MASH subject” has MASLD-Activity-Score (MAS)≥4, with at least 1 point in steatosis, at least 1 point in lobular inflammation, at least 1 point in hepatocyte ballooning and a liver fibrosis of stage 1, 2 or 3. In addition, in the context of the present invention, a patient is excluded as being a MASH patient if said patient has viral hepatitis, autoimmune liver disease, alcohol-related liver disease, drug-induced liver disease or congenital causes of chronic liver disease such as hereditary hemochromatosis, Wilson's disease, alpha-1-antitrypsin deficiency or polycystic ovary syndrome.

The MASLD-Activity-Score (MAS) is a score which has been developed to assess the severity of MASLD. MAS is the sum of three histological scores determined from liver biopsy slices:
S: Steatosis score: 0: <5%; 1:5-33%; 2:34-66% and 3: >66%;
LI: Lobular Inflammation score (foci per 20× field): 0: none; 1: <2 foci; 2:2-4 foci and 3: >4 foci; and
HB: Ballooning degeneration score: 0: none; 1: few; 2: many cells/prominent ballooning.

The term “steatosis” refers to the process describing the abnormal retention of lipids or fat accumulation within the liver. The term “hepatocellular ballooning” is usually defined, at the light microscopic level, based on hematoxylin and eosin (H&E) staining, as cellular enlargement 1.5-2 times the normal hepatocyte diameter, with rarefied cytoplasm. It refers more generally to the process of hepatocyte cell death. The term “lobular inflammation” refers to the presence of lobular inflammatory foci (grouped inflammatory cells) at microscopic examination of a hematoxylin and eosin (H&E) stained slice of a liver biopsy.

According to the present invention, the term “fibrosis” or “liver fibrosis” denotes a pathological condition of excessive deposition of fibrous connective tissue in the liver. More specifically, fibrosis is a pathological process which includes a persistent fibrotic scar formation and overproduction of extracellular matrix by the connective tissue, as a response to tissue damage. Physiologically, the deposit of connective tissue can obliterate the architecture and function of liver

The different stages of liver fibrosis are defined by the Kleiner scoring system (Kleiner et al, Hepatology 2005, Vol 41, Issue 6, 1313-1321) wherein:
F0 refers to a subject with an absence of liver fibrosis;
F1 refers to a subject with portal or perisinusoidal fibrosis;
F2 refers to a subject with portal/periportal and perisinusoidal fibrosis;
F3 refers to a subject with septal or bridging liver fibrosis;
F4 refers to a subject with liver cirrhosis.

Within the context of the present invention, “liver fibrosis improvement” or “the improvement of liver fibrosis” is defined as the improvement in liver fibrosis greater than or equal to one stage according to Kleiner scoring system and no worsening of steatohepatitis (defined as no increase in MAS for ballooning, inflammation, or steatosis) in a patient who had baseline MASH.

Within the context of the present invention, “MASH resolution” is defined as absent fatty liver disease or isolated or simple steatosis without steatohepatitis and a MAS score of 0-1 for inflammation, 0 for ballooning, and any value for steatosis, with no worsening of fibrosis score in a subject who had baseline MASH.

The term “subject who had baseline MASH” refers to a patient with MASH at the first timepoint of the method of the invention.

Subjects who had baseline MASH and achieved MASH resolution are defined as “MASH resolvers”.

Patients who had baseline MASH and achieved fibrosis improvement are defined as “fibrosis improvers”.

The terms “subject” and “patient” may be used interchangeably herein and refer to mammal subject, preferably a human subject.

In a particular embodiment, the computer program receives and stocks S1 values of a subject with MASH obtained at three or more timepoints.

In another particular embodiment, the computer program causes a step before step (a) to stock the information relating to the levels of hsa-miR-34a-5p and YKL-40 measured at three or more timepoints of a subject with MASH and the information relating to the gender of the subject and then calculates corresponding S1 values.

Within the scope of the invention, the term “timepoint” refers to a period of time during which the levels of hsa-miR-34a-5p and YKL-40 of a subject in said period of time are determined and stocked for the implementation of the computer program of the invention. A timepoint may be a period of time from several minutes to several days. For instance, said timepoint may be a period of 3 days. According to one embodiment, only one biological fluid sample of a subject is collected at one timepoint. According to said embodiment, the levels of hsa-miR-34a-5p and YKL-40 of a subject at a timepoint are obtained from the same sample. According to another embodiment, more than one, e.g. two or three, biological fluid samples are collected at one timepoint. According to a more particular embodiment, the respective levels of hsa-miR-34a-5p and YKL-40 of a subject at a timepoint are mean levels of those measured in all samples collected at one timepoint. According to another particular embodiment, the levels of hsa-miR-34a-5p and YKL-40 of a subject at a timepoint are obtained from different samples of a same subject.

In general, the first timepoint (T₀) occurs at the beginning of the follow-up of a subject, e.g. at the beginning of a treatment protocol of MASH and/or liver fibrosis or at the beginning of a life-style protocol or of a diet plan, just before the therapy has been initiated by the patient. The last timepoint (T_{End}) occurs at the end of the follow-up of a subject, e.g. at the end of a treatment protocol of MASH and/or liver fibrosis. The number of timepoints may be determined according to the time course of a treatment protocol or of a life-style protocol. In a particular embodiment, the computer program of the invention stocks or calculates S1 values from 3 to 10 timepoints. In a more particular embodiment, the computer program of the invention stocks or calculates S1 values from 8 timepoints. Two adjacent timepoints may be separated by a period of from several days to several months, for example from 2 weeks to 6 months. In a particular embodiment, two adjacent measurement timepoints are separated by a period of 9 to 12 weeks.

The information relating to the levels of hsa-miR-34a-5p and YKL-40 is based on the measure(s) performed samples of biological fluid of the patient. Such biological fluids include blood or a blood-derived sample, e.g. serum or plasma, or a sample of saliva, interstitial fluid or urine. In a particular embodiment, the sample is a sample of blood or a blood-derived sample, such as serum or plasma.

According to the present invention, a biological fluid sample may be analyzed immediately after collection or be stored in a cold environment, for example in a freezer, for further analysis.

The circulating level of YKL-40 may be measured by any conventional method well known in the art, such as immunoassays (e.g. ELISA (enzyme-linked immunosorbent assay), immunoturbidimetry, immuno-nephelometry, immune cytometry, protein array). For example, the levels of YKL-40 can be determined by antibodies, aptamers or peptides respectively directed against said markers from the serum. The level of YKL-40 can be measured using a YKL-40-specific binding agent, such as an anti-YKL-40 antibody. For example, one can cite methods based on immunodetection, such as ELISA (enzyme-linked immunosorbent assay). In this respect, commercial kits for YKL-40 level determination are readily available, such as the Quantikine® Chitinase 3-like 1 Immunoassay (ref.: DC3L10, R&D Systems, Inc., U.S.A.).

Circulating hsa-miR-34a-5p may be measured by common technique used in molecular biology such as RNA extraction, reverse transcription and quantitative PCR with specific primers and/or probes from the serum.

The circulating levels of hsa-miR-34a-5p and YKL-40 in a biological fluid sample may be simultaneously measured by an analytical method, such as biochips or immunoPCR.

According to the invention, the following equations can be used for calculating a S1 value.

Y=−1.4539+2.3003*log 10(miR-34a-5p(Fold))+1.0598*log 10(YKL-40 (ng/ml))−0.0533*Gender+0.4514*log 10(miR-34a-5p(Fold))*Gender equation 1:

Y=−0.8756+3.3957*log 10(miR-34a-5p(Fold))+2.5248*log 10(YKL-40 (ng/ml))−0.6496*Gender+0.2873*log 10(miR-34a-5p(Fold))*Gender equation 2:

Y=1.1543+2.5678*log 10(miR-34a-5p(Fold))+1.7859*log 10(YKL-40 (ng/ml))+0.3514*Gender+0.7264*log 10(miR-34a(5p(Fold))*Gender equation 3:

In step (b) of the computer program of the invention, corresponding S1 logit values are calculated.

In step (d) of the computer program of the invention, a total of 27 features, i.e. X1-X27, are calculated.

Features X1-X19 and X24-26 are time-series features:
X1: goodness of exponential fit to embedding distance distribution of S1 logit values, this feature indicating Mean Absolute Error (MAE) of an exponential fit to provided distribution of S1 logit values time-series,
X2: time reversibility of S1 logit values, this feature defining a property of some mathematical or physical processes and systems for which time-reversed dynamics are well defined (https://en.wikipedia.org/wiki/Time_reversibility),
X3: autocorrelation of differentiated time-series of S1 logit values, this feature defining how data points in a time series are related, on average, to the preceding data points (Box, Jenkins, & Reinsel, (1994) “Time Series Analysis; Forecasting and Control” 3rd Edition, Prentice-Hall, Englewood Cliffs),
X4: 5-bin histogram mode of S1 logit values, this feature meaning that a histogram with 5 bins (bars) is generated and the mode is selected,
X5: Proportion of S1 logit values inside a given circular boundary in a 2-d embedding space (https://pkg.robjhyndman.com/tsfeatures/reference/embed2_incircle.html; Fulcher and Jones, Cell Systems, 2017, 5 (5), 527-531; Fulcher et al., 2013, J. R. Soc. Interface. 1020130048),
X6: 10-bin histogram mode of S1 logit values, this feature meaning that a histogram with 10 bins (bars) is generated and the mode is selected,
X7: Ljung-box test statistic of S1 logit values, this feature examining the null hypothesis of independence in a given time-series,
X8: squared coefficient of variation of non-zero observations of S1 logit values (this feature corresponds to squared coefficient of variation: (SD/Mean)². “SD” being the standard deviation of S1 logit values at each timepoint, “Mean” being the mean of S1 logit values at all timepoints, the formula being squared. For example, when SD=1 and Mean=2, we obtain % CV (SD/Mean)=0.5 and % CV2=0.25),
X9: shanon entropy of S1 logit values, this feature quantifying the average amount of information contained in a random variable (https://en.wikipedia.org/wiki/Entropy_(information_theory)),
X10: strength of trend of S1 logit values, this features quantifying how prominent or significant the trend component is in a time-series, which can be calculated according to the method described in https://cran.r-project.org/web/packages/tsfeatures/vignettes/tsfeatures.html #ref,
X11: S1 scores percentage change between the last timepoint T_{End} and the first timepoint T₀,
X12: whether the S1 score at T_{End} is <0.46, value 1 being assigned if the answer is yes, 0 being assigned if the answer is no,
X13: whether the S1 scores of the subject reduce from ≥0.68 at T₀ to <0.46 at T_{End}, value 1 being assigned if the answer is yes, 0 being assigned if the answer is no,
X14: whether the S1 score of the subject progresses from <0.46 at T₀ to ≥0.68 at T_{End}, value 1 being assigned if the answer is yes, 0 being assigned if the answer is no,
X15: whether S1 stage increases between T₀ and T_{End}, value 1 being assigned if the answer is yes, 0 being assigned if the answer is no,
X16: S1 logit at T_{End},
X17: mean of the values of S1 logit at all timepoints (this feature relates to a mean of all of the S1 logit values, which are calculated at each timepoint. Features X17 is calculated by the following method:

$${\sum }_{T 0}^{T e n d} \frac{S 1 {Value}_{T n}}{number of timepoints} ) ,$$

X18: mean of all S1 logit scores delta between every two neighboring timepoints (this feature relates to a mean of all S1 logit scores delta between every two neighboring timepoints. As used herein, the term “S1 logit scores delta between every two neighboring timepoints” should be understood as the difference of values between a S1 logit score measured for the subject at the timepoint N+1 and a S1 logit score of the subject measured the timepoint N. Feature X18 is calculated by the following method:

$${\sum }_{T 0}^{T e n d - 1} \frac{( S 1 {value}_{T ( n + 1 )} - S 1 {value}_{T n} )}{number of timepoints} ) ,$$

X19: standard deviation of S1 logit values at each timepoint,
X24 centroid frequency of S1 logit values (https://en.wikipedia.org/wiki/Spectral_centroid), which indicates whether the time series slowly or quickly variates (https://time-series-features.gitbook.io/catch22-features/catch22/firstle_acf_tau),
X25: whether the S1 score at the first timepoint T₀ is <0.46, value 1 being assigned if the answer is yes, 0 being assigned if the answer is no,
X26: whether S1 stage decreases between T0 and TEnd, value 1 being assigned if the answer is yes, 0 being assigned if the answer is no.

In particular, features X1-X11, X16-19 and X24 may be calculated by well-established mathematical methods. In particular, features X1, X2, X3, X4, X5, X6, X7, X8, X9, X10 and X24 may be calculated by any software for the extraction of time-series features, e.g. the software package “theft” (Henderson and Fulcher, arXiv:2208.06146).

Within the scope of the invention for calculating the values of features X15 and X26, the S1 stage of a patient is determined by comparing a patient's S1 value with the reference S1 values, i.e. 0.46 and 0.68, which separate S1 scores into three stages, i.e. a first stage with S1<0.46, a second stage with S1 in the range [0.46, 0.68[ and a third stage with S1≥0.68. In other words, the S1 value of a patient changing either from <0.46 at T0 to ≥0.46 at TEnd or from [0.46, 0.68] at T0 to ≥0.68 at TEnd, means that the S1 stage of the patient is increased between T0 and TEnd.

In a particular embodiment, the values of features selected in the group consisting of X1, X2, X3, X4, X5, X6, X7, X8, X9, X10, X11, X16, X17, X18, X19, X21, X24, and X27 are standardized. More particularly, the standardized values of features X1, X2, X3, X4, X5, X6, X7, X8, X9, X10, X11, X16, X17, X18, X19, X21, X24 and X27 are used in the calculation of the S2 and S3 scores.

At least 19 features among the features X1-X23 are combined in a logistic function in step (e) to calculate a S2 score and at least 6 features among the features X2, X5, X24, X25, X26, X17, X22 and X27 are combined in a logistic function in step (f) to calculate a S3 score.

In a particular embodiment, S2 and S3 scores are calculated by a regression analysis method that performs both variable selection and regularization in order to enhance the prediction accuracy and interpretability of the resulting statistical model, e.g. the LASSO (least absolute shrinkage and selection operator) method.

In a further particular embodiment, S2 is calculated by combining the features X1-X23 in the following logistic function:

$$S 2 = \frac{{e}^{y}}{1 + {e}^{y}}$$

wherein

$$y = β0 + {\sum }_{n = 1}^{23} β n \times Xn$$

wherein:
β0 is comprised between −0.65215 and −0.5151;
β1 is comprised between 0.0058 and 0.0074;
β2 is comprised between −0.1184 and −0.0907;
β3 is comprised between −0.0565 and −0.0524;
β4 is comprised between 0.0709 and 0.0900;
β5 is comprised between 0.00068 and 0.00103;
β6 is comprised between 0.0156 and 0.0182;
β7 is comprised between −0.0799 and −0.0707;
β8 is comprised between −0.1201 and −0.1084;
β9 is comprised between −0.0961 and −0.0906;
β10 is comprised between 0.0687 and 0.1002;
β11 is comprised between −0.0153 and −0.0189;
β12 is comprised between 0.0324 and 0.0424;
β13 is comprised between 0.5457 and 0.6241;
β14 is comprised between −1.1548 and −0.9475;
β15 is comprised between −0.1768 and −0.1518;
β16 is comprised between −0.1338 and −0.1128;
β17 is comprised between −1.0343 and −0.7174;
β18 is comprised between 0.0463 and 0.0539;
β19 is comprised between −0.00129 and −0.00125;
β20 is comprised between 0.3786 and 0.4439;
β21 is comprised between −0.3022 and −0.2804;
β22 is comprised between −0.4213 and −0.2989;
β23 is comprised between 0.1744 and 0.2377.

In a more particular embodiment, S2 is calculated with the following coefficients:
β0 is comprised between −0.6339 and −0.5380;
β1 is comprised between 0.0059 and 0.0068;
β2 is comprised between −0.1117 and −0.1069;
β3 is comprised between −0.0546 and −0.0527;
β4 is comprised between −0.0815 and −0.0865;
β5 is comprised between 0.00075 and 0.00097;
β6 is comprised between 0.0159 and 0.0170;
β7 is comprised between −0.0794 and −0.0715;
β8 is comprised between −0.1156 and −0.1085;
β9 is comprised between −0.0954 and −0.0933;
β10 is comprised between 0.0785 and 0.0881;
β11 is comprised between −0.0161 and −0.0180;
β12 is comprised between 0.0375 and 0.0395;
β13 is comprised between 0.5658 and 0.6100;
β14 is comprised between −1.1251 and −0.9651;
β15 is comprised between −0.1719 and −0.1631;
β16 is comprised between −0.1331 and −0.1155;
β17 is comprised between −0.9747 and −0.8239;
β18 is comprised between 0.0466 and 0.0529;
β19 is comprised between −0.00129 and −0.00127;
β20 is comprised between 0.3993 and 0.4316;
β21 is comprised between −0.2969 and −0.2817;
β22 is comprised between −0.3665 and −0.3542;
β23 is comprised between 0.1937 and 0.2235.

In another particular embodiment, S3 is calculated by combining the 8 features X2, X5, X24, X25, X26, X17, X27 and X22 in the following logistic function:

$$S 3 = \frac{{e}^{y}}{1 + {e}^{y}}$$

wherein y=P0+P1×X2+P2×X5+P3×X24+P4×X25+P5×X26+P6×X17+P7×X27+P8×X22,
wherein:
P0 is comprised between −0.0220 and −0.0196;
P1 is comprised between −0.1459 and −0.1154;
P2 is comprised between −0.0475 and −0.0351;
P3 is comprised between −0.0643 and −0.0556;
P4 is comprised between −0.1585 and −0.1109;
P5 is comprised between 0.3121 and 0.4023;
P6 is comprised between −0.5694 and −0.4475;
P7 is comprised between −0.2426 and −0.2124;
P8 is comprised between −0.6328 and −0.4951.

In a more particular embodiment of the above logic function, S3 is calculated with the following coefficients:
P0 is comprised between −0.0200 and −0.0198;
P1 is comprised between −0.1418 and −0.1354;
P2 is comprised between −0.0469 and −0.0427;
P3 is comprised between −0.0627 and −0.0568;
P4 is comprised between −0.1490 and −0.1281;
P5 is comprised between 0.3354 and 0.3927;
P6 is comprised between −0.5274 and −0.4683;
P7 is comprised between −0.2404 and −0.2284;
P8 is comprised between −0.5858 and −0.5126.

In a particular embodiment, the reference S2 value is comprised between 0.4160 and 0.7238. In a more particular embodiment, the reference S2 value is comprised between 0.4210 and 0.7076.

In another particular embodiment, the reference S3 value is comprised between 0.5054 and 0.5954. In a more particular embodiment, the reference S3 value is comprised between 0.5122 and 0.5844.

After the comparison of the S2 and S3 values of a subject with MASH with reference S2 and S3 scores performed in step (g) of the computer program of the invention, following information is provided as the output of the computer program:
the subject achieved MASH resolution and liver fibrosis improvement and/or the treatment of MASH and liver fibrosis in the subject is efficient, when the subject's S2 score is higher than the reference S2 value and the subject's S3 score is higher than the reference S3 value;
the subject achieved liver fibrosis improvements and that the subject does not achieve MASH resolution, when the subject's S2 score is lower than the reference S2 value and the subject's S3 score is higher than the reference S3 value;
the subject achieved MASH resolution and that the subject does not achieve liver fibrosis improvement, when the subject's S2 score is higher than the reference S2 value and the subject's S3 score is lower than the reference S3;
the subject achieved neither MASH resolution nor liver fibrosis improvement and/or the treatment of MASH and liver fibrosis in the subject is not efficient, when the subject's S2 score is lower than the reference S2 value and the subject's S3 score is lower than the reference S3 score value.

According to another aspect, the present invention further provides a computer-readable medium comprising the computer program described therein. According to a particular embodiment, the computer readable medium is a non-transitory computer-readable medium or a storage medium. In a particular embodiment, the storage medium is a RAM, a CD-ROM or a disk drive, such as a hard drive or a solid-state drive.

According to another aspect, the invention relates to a system for evaluating the efficacy of a treatment of metabolic dysfunction-associated steatohepatitis (MASH) and/or for predicting the resolution of MASH and the improvement of liver fibrosis in a subject with MASH with the aid of a digital computer, for use in determining whether said subject with MASH achieves MASH resolution and/or liver fibrosis improvement, said system comprising:
a database;
at least one central processing unit; and
a memory comprising the computer program described herein.

In a particular embodiment, the database stores patients' data such as the levels of the markers discussed above, the age and the gender of the patients.

The invention will now be further illustrated with reference to the following examples, which should not be interpreted as limiting the scope of the invention to the specific methods and features disclosed therein.

This disclosure also provides the following non-limiting embodiments”
1. An invention as shown in FIG. 1 and described herein.
2. A computer program comprising instructions that, when executed by a central processing unit (CPU), cause the central processing unit to evaluate the efficacy of a treatment of metabolic dysfunction-associated steatohepatitis (MASH) and/or to predict the resolution of MASH and the improvement of liver fibrosis in a subject with MASH,
wherein the program causes the CPU to:
   a) stock or calculate S1 values obtained at three or more timepoints of a subject with MASH,
   b) calculate the corresponding S1 value logits;
   c) stock the information relating to Type-2 diabetes comorbidity at T₀, dyslipidemia comorbidity at T₀, waist circumference of the subject at T₀ and BMI index at T₀;
   d) calculate the values of 27 features X1-X27;
   e) combine the values of at least 19 features among the features X1-X23 in a mathematical function to calculate a S2 score for the subject,
   f) combine the values at least 6 features among the features X2, X5, X24, X25, X26, X17, X22 and X27 in a mathematical function to calculate a S3 score for the subject,
   g) compare said S2 score with a reference S2 value and said S3 score with a reference S3 value, wherein:
      the subject's S2 score higher than the reference S2 value and the subject's S3 score higher than the reference S3 value indicate that the subject achieved MASH resolution and liver fibrosis improvement and/or the treatment of MASH and liver fibrosis in the subject is efficient;
      the subject's S2 score lower than the reference S2 value and the subject's S3 score higher than the reference S3 value indicate that the subject achieved liver fibrosis improvements and that the subject does not achieve MASH resolution;
      the subject's S2 score higher than the reference S2 value and the subject's S3 score lower than the reference S3 indicate that the subject achieved MASH resolution and that the subject does not achieve liver fibrosis improvement;
      the subject's S2 score lower than the reference S2 value and the subject's S3 score lower than the reference S3 score value indicate that the subject achieved neither MASH resolution nor liver fibrosis improvement and/or the treatment of MASH and liver fibrosis in the subject is not efficient,
wherein the features X1-X27 are:
   X1: goodness of exponential fit to embedding distance distribution of S1 logit values,
   X2: time reversibility of S1 logit values,
   X3: autocorrelation of differentiated time-series of S1 logit values
   X4: 5-bin histogram mode of S1 logit values,
   X5: Proportion of S1 logit values inside a given circular boundary in a 2-d embedding space,
   X6: 10-bin histogram mode of S1 logit values,
   X7: Ljung-box test statistic of S1 logit values,
   X8: squared coefficient of variation of non-zero observations of S1 logit values,
   X9: shanon entropy of S1 logit values,
   X10: strength of trend of S1 logit values,
   X11: S1 scores percentage change between the last timepoint T_{End} and the first timepoint T₀
   X12: whether the S1 score at T_{End} is <0.46, value 1 being assigned if the answer is yes, 0 being assigned if the answer is no,
   X13: whether the S1 scores of the subject reduce from ≥0.68 at T₀ to <0.46 at T_{End}, value 1 being assigned if the answer is yes, 0 being assigned if the answer is no,
   X14: whether the S1 score of the subject progresses from <0.46 at T₀ to ≥0.68 at T_{End}, value 1 being assigned if the answer is yes, 0 being assigned if the answer is no,
   X15: whether S1 stage is increased between T₀ and T_{End}, value 1 being assigned if the answer is yes, 0 being assigned if the answer is no,
   X16: S1 logit at T_{End},
   X17: mean of the values of S1 logit at all timepoints,
   X18: mean of all S1 logit scores delta between every two neighboring points,
   X19: standard deviation of S1 logit values at each timepoint,
   X20: whether the gender of the subject is male, value 1 being assigned if the answer is yes, 0 being assigned if the answer is no,
   X21: the body mass index (BMI) of the subject at T₀,
   X22: whether the subject has Type-2 diabetes comorbidity at T₀, value 1 being assigned if the answer is yes, 0 being assigned if the answer is no,
   X23: whether the subject has dyslipidemia comorbidity, value 1 is assigned if the answer is yes, 0 being assigned if the answer is no,
   X24: Centroid frequency of S1 logit values,
   X25: whether the S1 score at the first timepoint T₀ is <0.46, value 1 being assigned if the answer is yes, 0 being assigned if the answer is no,
   X26: whether S1 stage decreases between T₀ and T_{End}, value 1 being assigned if the answer is yes, 0 being assigned if the answer is no,
   X27: waist circumference of the subject at T₀,
wherein the score S1 is obtained from a logistic function:

$$S 1 = \frac{1}{1 + exp ( - Y )}$$

wherein

Y=B0+B1*log 10(miR-34a-5p(Fold))+B2*log 10(YKL-40 (ng/ml))+B3*Gender+B4*log 10(miR-34a-5p(Fold))*Gender
wherein
Gender is 0 if the subject is a female, or 1 if the subject is a male;
B0 is comprised between −3 and 3, in particular between −2 and 2;
B1 is comprised between 1 and 5, in particular between 2 and 4;
B2 is comprised between 0 and 4.5, in particular between 0.5 and 3;
B3 is comprised between −2 and 2, in particular between −1 and 1; and
B4 is comprised between −1 and 2, in particular between 0 and 2.
3. The computer program according to embodiment 2, wherein the computer program causes a step before step (a) to stock the information relating to the levels of hsa-miR-34a-5p and YKL-40 measured at three or more timepoints of a subject with MASH and the information relating to the gender of the subject.
4. The computer program according to embodiment 2 or 3, wherein the values of features selected in the group consisting of X1, X2, X3, X4, X5, X6, X7, X8, X9, X10, X11, X16, X17, X18, X19, X21, X24, and X27 are standardized.
5. The computer program according to embodiment 2, wherein the score S2 is obtained by combining the values of 23 features in a logistic function:

$$S 2 = \frac{{e}^{y}}{1 + {e}^{y}}$$

Wherein

$$y = β0 + {\sum }_{n = 1}^{23} β n \times Xn$$

β0 is comprised between −0.65215 and −0.5151, in particular between −0.6339 and −0.5380;
β1 is comprised between 0.0058 and 0.0074, in particular between 0.0059 and 0.0068;
β2 is comprised between −0.1184 and −0.0907, in particular between −0.1117 and −0.1069;
β3 is comprised between −0.0565 and −0.0524, in particular between −0.0546 and −0.0527;
β4 is comprised between 0.0709 and 0.0900, in particular between 0.0815 and 0.0865;
β5 is comprised between 0.00068 and 0.00103, in particular between 0.00075 and 0.00097;
β6 is comprised between 0.0156 and 0.0182, in particular between 0.0159 and 0.0170;
β7 is comprised between −0.0799 and −0.0707, in particular between −0.0794 and −0.0715;
β8 is comprised between −0.1201 and −0.1084, in particular between −0.1156 and −0.1085;
β9 is comprised between −0.0961 and −0.0906, in particular between −0.0954 and −0.0933;
β10 is comprised between 0.0687 and 0.1002, in particular between 0.0785 and 0.0881;
β11 is comprised between 0.0153 and 0.0189, in particular between 0.0161 and 0.0180;
β12 is comprised between 0.0324 and 0.0424, in particular between 0.0375 and 0.0395;
β13 is comprised between 0.5457 and 0.6241, in particular between 0.5658 and 0.6100;
β14 is comprised between −1.1548 and −0.9475, in particular between −1.1251 and −0.9651;
β15 is comprised between −0.1768 and −0.1518, in particular between −0.1719 and −0.1631;
β16 is comprised between −0.1338 and −0.1128, in particular between −0.1331 and −0.1155;
β17 is comprised between −1.0343 and −0.7174, in particular between −0.9747 and −0.8239;
β18 is comprised between 0.0463 and 0.0539, in particular between 0.0466 and 0.0529;
β19 is comprised between −0.00129 and −0.00125, in particular between −0.00129 and −0.00127;
β20 is comprised between 0.3786 and 0.4439, in particular between 0.3993 and 0.4316;
β21 is comprised between −0.3022 and −0.2804, in particular between −0.2969 and −0.2817;
β22 is comprised between −0.4213 and −0.2989, in particular between −0.3665 and −0.3542;
β23 is comprised between 0.1744 and 0.2377, in particular between 0.1937 and 0.2235;
wherein the score S3 is obtained by combining the values of 8 features in a logistic function:

$$S 3 = \frac{{e}^{y}}{1 + {e}^{y}}$$

Wherein y=P0+P1×X2+P2×X5+P3×X24+P4×X25+P5×X26+P6×X17+P7× X27+P8×X22
Wherein:
P0 is comprised between −0.0220 and −0.0196, in particular between −0.0200 and −0.0198;
P1 is comprised between −0.1459 and −0.1154, in particular between −0.1418 and −0.1354;
P2 is comprised between −0.0475 and −0.0351, in particular between −0.0469 and −0.0427;
P3 is comprised between −0.0643 and −0.0556, in particular between −0.0627 and −0.0568;
P4 is comprised between −0.1585 and −0.1109, in particular between −0.1490 and −0.1281;
P5 is comprised between 0.3121 and 0.4023, in particular between 0.3354 and 0.3927;
P6 is comprised between −0.5694 and −0.4475, in particular between −0.5274 and −0.4683;
P7 is comprised between −0.2426 and −0.2124, in particular between −0.2404 and −0.2284;
P8 is comprised between −0.6328 and −0.4951, in particular between −0.5858 and −0.5126.
6. The computer program according to embodiment 5, wherein the reference S2 score is comprised between 0.4160 and 0.7238.
7. The computer program according to embodiment 6, wherein the reference S2 score is comprised between 0.4210 and 0.7076.
8. The computer program according to embodiment 5, wherein the reference S3 score is comprised between 0.5054 and 0.5954.
9. The computer program according to embodiment 8, wherein the reference S3 score is comprised between 0.5122 and 0.5844.
10. A computer readable medium comprising the computer program according to any one of embodiments 2-9.
11. A system for evaluating the efficacy of a treatment of metabolic dysfunction-associated steatohepatitis (MASH) and/or for predicting the resolution of MASH and the improvement of liver fibrosis in a subject with MASH with the aid of a digital computer, for use in determining whether said subject with MASH achieves MASH resolution and/or liver fibrosis improvement, said system comprising:
   a database;
   at least one central processing unit; and
   a memory comprising the computer program according to any one of embodiments 2 to 9.

### EXAMPLES

### 1. Patient Selection

The patients used for the analyses correspond to patients included in Resolve-IT phase 3 clinical trial, who had a liver-biopsy proven fibrosis stage of 1, 2, or 3; a MAS score of 4 or more points; a ballooning score of 1 point or more; a lobular inflammation score of 1 point or more; a steatosis of 1 point or more. Selected patients for the analysis had available liver biopsy at screening visit (SV) and visit 7, had less than 180 days between each liver biopsy and their blood dosage (at screening visit and visit 7), and had available demography information (sex, age, BMI, waist, comorbidities information: type-2 diabetes, hypertension, dyslipidemia) at screening visit (baseline).

At each timepoint, a S1 value is calculated based on the measured levels of circulating hsa-miR-34a-5p and YKL-40 and the gender of a patient and according to the logistic function below:

$$S 1 = \frac{1}{1 + exp ( - y )}$$

wherein

Y=−0.8756+3.3957*log 10(miR-34a-5p(Fold))+2.5248*log 10(YKL-40 (ng/ml))−0.6496*Gender+0.2873*log 10(miR-34a-5p(Fold))*Gender

Two cohorts were created. Cohort A patients with S1 values available at every study visit (Screening visit, and visits 1-2-3-4-5-6-7), this selection led to 423 patients used for the analyses. Cohort B with S1 values available at the following visits (Screening visit, visits 1-3-5-7) and who were not part of cohort A, this selection led to 234 patients.

### Cohort Descriptive

The physiological, demographical and histological features of the cohort are detailed in Table 1.

TABLE 1

| | 8 Visits (N = 423) | 5 Visits (N = 234) |
|---|---|---|
| | (SV-V1-V2-V3-V4-V5-V6-V7) | (SV-V1-V3-V5-V7) |
| Demographics | | |
| Age | 53.89 ± 11.46 | 54.91 ± 12.74 |
| Sex (male) | 52.96 (224) | 47.44 (111) |
| Clinical characteristics | | |
| BMI (kg/m2) | 33.95 ± 6.25 | 33.91 ± 6.04 |
| Type-2 diabetes | 47.52 (201) | 44.87 (105) |
| Hypertension | 60.28 (255) | 57.26 (134) |
| Dyslipidemia | 51.3 (217) | 54.27 (127) |
| Treatment | 65.72 (278) | 68.8 (161) |
| Laboratory tests | | |
| Alat (ui/l) | 70.47 ± 49.00 | 69.4 ± 47.29 |
| Asat (ui/l) | 49.77 ± 31.94 | 49.41 ± 27.25 |
| GGT (ui/l) | 76.24 ± 77.49 | 78.44 ± 81 |
| Alkaine phosphatase (ui/l) | 85.91 ± 29.13 | 85.26 ± 30.77 |
| Fasting plasma glucose | 6.17 ± 1.71 | 6.27 ± 1.91 |
| (mmol/l) | | |
| HbA1c (%) | 6.30 ± 1.01 | 6.23 ± 0.99 |
| Triglycerides (mmol/l) | 2.11 ± 1.27 | 2.06 ± 1.08 |
| Platelet (10\hat{ }9/1) | 242.69 ± 66.65 | 240.43 ± 63.64 |
| Histology | | |
| Baseline | | |
| Fibrosis stage | 0/13.9/40.4/45.6/0 | 0/14.1/43.2/42.7/0 |
| (baseline), 0/1/2/3/4 | | |
| MAS score (baseline), | 0/0/40.9/59.1 | 0/0/47/53 |
| 0-1/2-3/4-5/6-8 | | |
| Ballooning grade | 0/33.6/66.4 | 0/31.2/68.8 |
| (baseline), 0/1/2 | | |
| Lobular inflammation | 0/54.1/40.2/5.7 | 0/62.8/33.8/3.4 |
| (baseline), 0/1/2/3 | | |
| Steatosis grade | 0/7.1/31.4/61.5 | 0/6.4/31.2/62.4 |
| (baseline), 0/1/2/3 | | |
| Histological endpoints* | | |
| MASH resolution | 20.8 (88) | 21.8 (51) |
| Fibrosis improvement | 22.2 (94) | 17.5 (41) |
| Composite endpoint | 10.6 (45) | 11.5 (27) |

As indicated in table 1, at baseline, all patients were MASH (steatosis ≥1, lobular inflammation ≥1 & ballooning ≥1), with fibrosis 1-3 & MAS 4-8.

MASH resolution prevalence was 21-23% in different cohorts; 18-23% for fibrosis improvement; and 11-13% for composite endpoint. These low prevalences highlight the fact that it will be complex to reach elevated PPV values, the latter being directly linked to prevalence.

### 2. Performance of S2 and S3 Scores

Overall performances for predicting MASH resolution and liver fibrosis improvement

AUC of S2 score and AUC of S3 score were calculated in cohort A and cohort B (Table 2). These results show that S2 and S3 scores provide a good performance for predicting MASH resolution and liver fibrosis respectively.

TABLE 2

| | Cohort A | Cohort B |
|---|---|---|
| | (8 visits) | (5 visits) |
| score | AUC | AUC |
| S2 for MASH resolution | 0.811 | 0.802 |
| | (0.760-0.862) | (0.736-0.867) |
| S3 for fibrosis | 0.741 | 0.772 |
| improvement | (0.687-0.796) | (0.687-0.858) |

### 3. Performance of S2 and S3 Scores for Predicting MASH Resolution and Liver Fibrosis Improvement

Demographical features of four patients wish MASH (patients A, B, C and D) were registered at inclusion visit (baseline timepoint). The levels of hsa-miR-34a-5p and YKL-40 in blood samples of these patients collected during different visits. S1 values at these timepoints were also registered and S1 logit value at each timepoint was calculated by implementing the computer program of the invention. The input information and the calculated S1 logit values are detailed in Table 3 below.

TABLE 3

| inclusion_{—} | visit_{—} | | | sex_{—} | bmi_{—} | waist_cm_{—} | diab2_{—} | dyslipidemia_{—} |
|---|---|---|---|---|---|---|---|---|
| numb | rank | S1 | logitS1 | SV | SV | SV | SV | SV |
| A | 1 | 0.5 | 0 | 0 | 36 | 111 | 1 | 1 |
| A | 2 | 0.57 | 0.28185115 | 0 | 36 | 111 | 1 | 1 |
| A | 3 | 0.72 | 0.94446161 | 0 | 36 | 111 | 1 | 1 |
| A | 4 | 0.7 | 0.84729786 | 0 | 36 | 111 | 1 | 1 |
| A | 5 | 0.77 | 1.20831121 | 0 | 36 | 111 | 1 | 1 |
| A | 6 | 0.78 | 1.26566637 | 0 | 36 | 111 | 1 | 1 |
| A | 7 | 0.76 | 1.15267951 | 0 | 36 | 111 | 1 | 1 |
| A | 8 | 0.85 | 1.73460106 | 0 | 36 | 111 | 1 | 1 |
| B | 1 | 0.72 | 0.94446161 | 1 | 28 | 89 | 0 | 1 |
| B | 2 | 0.65 | 0.61903921 | 1 | 28 | 89 | 0 | 1 |
| B | 3 | 0.55 | 0.2006707 | 1 | 28 | 89 | 0 | 1 |
| B | 4 | 0.57 | 0.28185115 | 1 | 28 | 89 | 0 | 1 |
| B | 5 | 0.32 | −0.7537718 | 1 | 28 | 89 | 0 | 1 |
| B | 6 | 0.22 | −1.2656664 | 1 | 28 | 89 | 0 | 1 |
| B | 7 | 0.24 | −1.1526795 | 1 | 28 | 89 | 0 | 1 |
| C | 1 | 0.3 | −0.8472979 | 0 | 31 | 77 | 0 | 0 |
| C | 2 | 0.35 | −0.6190392 | 0 | 31 | 77 | 0 | 0 |
| C | 3 | 0.32 | −0.7537718 | 0 | 31 | 77 | 0 | 0 |
| C | 4 | 0.37 | −0.5322168 | 0 | 31 | 77 | 0 | 0 |
| C | 5 | 0.29 | −0.895384 | 0 | 31 | 77 | 0 | 0 |
| D | 1 | 0.73 | 0.99462258 | 1 | 34 | 145 | 1 | 1 |
| D | 2 | 0.71 | 0.89538405 | 1 | 34 | 145 | 1 | 1 |
| D | 3 | 0.79 | 1.32492541 | 1 | 34 | 145 | 1 | 1 |

27 features (X1-X27) as defined above were calculated by the computer program of the invention for each patient from the data of table 3. The values of these features are detailed in table 4. These features are then standardized by implementing the computer program of the invention and detailed in table 5.

TABLE 4

| Patient number | A | B | C | D |
|---|---|---|---|---|
| NIS2CO_Embed2_Dist_tau_d_expfit_meandiff (X1) | 0.13217987 | 0.09425626 | 0.33613339 | 0.77873312 |
| NIS2CO_trev_1_num (X2) | 0.4566767 | −0.3233791 | −1.94133075 | 3.44099121 |
| NIS2diff1_acf1 (X3) | −0.41401875 | −0.32213333 | −0.55920482 | −0.5 |
| NIS2DN_HistogramMode_5 (X4) | 0.51055336 | 0.16655789 | −0.84898082 | 0.04390863 |
| NIS2embed2_incircle (X5) | 0.25 | 0 | 0.25 | 0.5 |
| NIS2histogram_mode (X6) | 0.25 | 0.5 | −1.1 | −0.7 |
| NIS21b_stat (X7) | 2.02201277 | 3.68664958 | 2.85637122 | 0.70751011 |
| NIS2nonzero_squared_cv (X8) | 0.17611297 | 30.3492736 | 0.04373342 | 0.04403932 |
| NIS2spectral_entropy (X9) | 0.52116843 | 0.28693565 | 0.61804899 | 1 |
| NIS2trend_strength (X10) | 0.89384595 | 0.92063203 | 9.3896E−05 | 0.53929073 |
| NIS2PercChange (X11) | −70 | 66.6666667 | 3.33333333 | −8.21917808 |
| NIS2coEOS_lc1 (X12) | 0 | 1 | 1 | 0 |
| NIS2coEvo_hcTolc1 (X13) | 0 | 1 | 0 | 0 |
| NIS2coEvo_lcTohc1 (X14) | 0 | 0 | 0 | 0 |
| NIS2coInc1 (X15) | 1 | 0 | 0 | 0 |
| logitNIS2EOS (X16) | 1.73460106 | −1.15267951 | −0.89538405 | 1.32492541 |
| logitNIS2Mean (X17) | 0.9293586 | −0.16087072 | −0.72954195 | 1.07164401 |
| logitNIS2MeanDelta (X18) | 0.24780015 | −0.34952352 | −0.01202155 | 0.16515142 |
| logitNIS2SD (X19) | 0.55794783 | 0.88623959 | 0.15256573 | 0.22489036 |
| demo_sex_SV1 (X20) | 0 | 1 | 0 | 1 |
| vitl_bmi_SV (X21) | 36 | 28 | 31 | 34 |
| medh_diab2_SV1 (X22) | 1 | 0 | 0 | 1 |
| medh_dyslipidemia_SV1 (X23) | 1 | 1 | 0 | 1 |
| NIS2SP_Summaries_welch_rect_centroid (X24) | 1.57079633 | 0.78539816 | 3.14159265 | 1.57079633 |
| NIS2coBaseline_lc1 (X25) | 0 | 0 | 1 | 0 |
| NIS2coDeac1 (X26) | 0 | 0 | 0 | 0 |
| vitl_waist_cm_SV (X27) | 111 | 89 | 77 | 145 |
| NIS2CO_Embed2_Dist_tau_d_expfit_meandiff | −1.95093987 | −2.54614896 | 1.25009946 | 8.19667815 |
| NIS2CO_trev_1_num | 0.40399864 | −0.11476467 | −1.19075685 | 2.38866803 |
| NIS2diff1_acf1 | −0.47075876 | −0.12613486 | −1.01529109 | −0.79323853 |
| NIS2DN_HistogramMode_5 | 0.96323342 | 0.3261154 | −1.55477569 | 0.09895527 |
| NIS2embed2_incircle | −0.734044 | −2.09527106 | −0.734044 | 0.62718305 |
| NIS2histogram_mode | 0.90648993 | 1.2874824 | −1.15086939 | −0.54128144 |
| NIS21b_stat | 0.76094602 | 2.11291293 | 1.43858634 | −0.30665269 |
| NIS2nonzero_squared_cv | −0.11654324 | 0.05956296 | −0.11731587 | −0.11731409 |
| NIS2spectral_entropy | 0.01012298 | −0.90681987 | 0.38937789 | 1.88458807 |
| NIS2trend_strength | 2.18720746 | 2.30203515 | −1.64416632 | 0.66728526 |
| NIS2PercChange | −1.54951991 | 1.42298904 | 0.04548489 | −0.20578299 |
| NIS2coEOS_lc1 | 0 | 1 | 1 | 0 |
| NIS2coEvo_hcTolc1 | 0 | 1 | 0 | 0 |
| NIS2coEvo_lcTohc1 | 0 | 0 | 0 | 0 |
| NIS2coInc1 | 1 | 0 | 0 | 0 |
| logitNIS2EOS | 0.80360623 | −1.45685773 | −1.25542004 | 0.48286947 |
| logitNIS2Mean | 0.10908808 | −0.98942656 | −1.56241948 | 0.25245481 |
| logitNIS2MeanDelta | 1.56782879 | −2.23767016 | −0.08747343 | 1.04128066 |
| logitNIS2SD | −0.67145073 | 0.49697107 | −2.11424442 | −1.85683415 |
| demo_sex_SV1 | 0 | 1 | 0 | 1 |
| vitl_bmi_SV | 0.42461273 | −0.98171602 | −0.45434274 | 0.07303055 |
| medh_diab2_SV1 | 1 | 0 | 0 | 1 |
| medh_dyslipidemia_SV1 | 1 | 1 | 0 | 1 |
| NIS2SP_Summaries_welch_rect_centroid | 0.15800066 | −1.10600463 | 2.68601124 | 0.15800066 |
| NIS2coBaseline_lc1 | 0 | 0 | 1 | 0 |
| NIS2coDeac1 | 0 | 0 | 0 | 0 |
| vitl_waist_cm_SV | 0.08661504 | −1.60719014 | −2.53108387 | 2.70431395 |

S2 scores of each of these four patients were calculated from the standardized features values and compared with the reference S2 value (0.6831) by implementing the computer program of the invention.

S3 scores of each of these four patients were calculated from the standardized features values and compared with the reference S3 value (0.5679) by implementing the computer program of the invention.

The results of predictions for the four patients based on their S2 and S3 scores were compared with the biopsy results of these patients at the end of the clinical trial. (Table 6).

TABLE 6

| | | | MASH | Liver fibrosis | |
|---|---|---|---|---|---|
| | | | resolution | improvement | Biopsy results at |
| | S2 score of | S3 score of | 1 (yes); 0 | 1 (yes); 0 | the end of clinical |
| Patients | patient | patient | (non) | (non) | trial |
| A | 0.34870728 | 0.468215982 | 0 | 0 | MASH not |
| | | | | | resolved and no |
| | | | | | liver fibrosis |
| | | | | | improvement |
| B | 0.69219407 | 0.736868659 | 1 | 1 | MASH resolved |
| | | | | | and liver fibrosis |
| | | | | | improved |
| C | 0.69041435 | 0.80012762 | 1 | 1 | MASH resolved |
| | | | | | and liver fibrosis |
| | | | | | improved |
| D | 0.25687861 | 0.240748281 | 0 | 0 | MASH not |
| | | | | | resolved and no |
| | | | | | liver fibrosis |
| | | | | | improvement |

According to these results, patients B and C achieved MASH resolution and liver fibrosis improvement, while patients A and D achieved neither MASH resolution nor liver fibrosis improvement. These predictions were in line with the biopsy results of these patients at the end of the clinical trial.

1. An invention as shown in FIG. 1 and described herein.

2. A computer program comprising instructions that, when executed by a central processing unit (CPU), cause the central processing unit to evaluate the efficacy of a treatment of metabolic dysfunction-associated steatohepatitis (MASH) and/or to predict the resolution of MASH and the improvement of liver fibrosis in a subject with MASH,
wherein the program causes the CPU to:
a) stock or calculate S1 values obtained at three or more timepoints of a subject with MASH,
b) calculate the corresponding S1 value logits;
c) stock the information relating to Type-2 diabetes comorbidity at T₀, dyslipidemia comorbidity at T₀, waist circumference of the subject at T₀ and BMI index at T₀;
d) calculate the values of 27 features X1-X27;
e) combine the values of at least 19 features among the features X1-X23 in a mathematical function to calculate a S2 score for the subject,
f) combine the values at least 6 features among the features X2, X5, X24, X25, X26, X17, X22 and X27 in a mathematical function to calculate a S3 score for the subject,
g) compare said S2 score with a reference S2 value and said S3 score with a reference S3 value, wherein:
the subject's S2 score higher than the reference S2 value and the subject's S3 score higher than the reference S3 value indicate that the subject achieved MASH resolution and liver fibrosis improvement and/or the treatment of MASH and liver fibrosis in the subject is efficient;
the subject's S2 score lower than the reference S2 value and the subject's S3 score higher than the reference S3 value indicate that the subject achieved liver fibrosis improvements and that the subject does not achieve MASH resolution;
the subject's S2 score higher than the reference S2 value and the subject's S3 score lower than the reference S3 indicate that the subject achieved MASH resolution and that the subject does not achieve liver fibrosis improvement;
the subject's S2 score lower than the reference S2 value and the subject's S3 score lower than the reference S3 score value indicate that the subject achieved neither MASH resolution nor liver fibrosis improvement and/or the treatment of MASH and liver fibrosis in the subject is not efficient,
wherein the features X1-X27 are:
X1: goodness of exponential fit to embedding distance distribution of S1 logit values,
X2: time reversibility of S1 logit values,
X3: autocorrelation of differentiated time-series of S1 logit values
X4: 5-bin histogram mode of S1 logit values,
X5: Proportion of S1 logit values inside a given circular boundary in a 2-d embedding space,
X6: 10-bin histogram mode of S1 logit values,
X7: Ljung-box test statistic of S1 logit values,
X8: squared coefficient of variation of non-zero observations of S1 logit values,
X9: shanon entropy of S1 logit values,
X10: strength of trend of S1 logit values,
X11: S1 scores percentage change between the last timepoint T_{End} and the first timepoint T₀
X12: whether the S1 score at T_{End} is <0.46, value 1 being assigned if the answer is yes, 0 being assigned if the answer is no,
X13: whether the S1 scores of the subject reduce from ≥0.68 at T₀ to <0.46 at T_{End}, value 1 being assigned if the answer is yes, 0 being assigned if the answer is no,
X14: whether the S1 score of the subject progresses from <0.46 at T₀ to ≥0.68 at T_{End}, value 1 being assigned if the answer is yes, 0 being assigned if the answer is no,
X15: whether S1 stage is increased between T₀ and T_{End}, value 1 being assigned if the answer is yes, 0 being assigned if the answer is no,
X16: S1 logit at T_{End},
X17: mean of the values of S1 logit at all timepoints,
X18: mean of all S1 logit scores delta between every two neighboring points,
X19: standard deviation of S1 logit values at each timepoint,
X20: whether the gender of the subject is male, value 1 being assigned if the answer is yes, 0 being assigned if the answer is no,
X21: the body mass index (BMI) of the subject at T₀,
X22: whether the subject has Type-2 diabetes comorbidity at T₀, value 1 being assigned if the answer is yes, 0 being assigned if the answer is no,
X23: whether the subject has dyslipidemia comorbidity, value 1 is assigned if the answer is yes, 0 being assigned if the answer is no,
X24: Centroid frequency of S1 logit values,
X25: whether the S1 score at the first timepoint T₀ is <0.46, value 1 being assigned if the answer is yes, 0 being assigned if the answer is no,
X26: whether S1 stage decreases between T₀ and T_{End}, value 1 being assigned if the answer is yes, 0 being assigned if the answer is no,
X27: waist circumference of the subject at T₀,
wherein the score S1 is obtained from a logistic function:
$$S 1 = \frac{1}{1 + exp ( - Y )}$$
wherein
Y=B0+B1*log 10(miR-34a-5p(Fold))+B2*log 10(YKL-40 (ng/ml))+B3*Gender+B4*log 10(miR-34a-5p(Fold))*Gender
wherein
Gender is 0 if the subject is a female, or 1 if the subject is a male;
B0 is comprised between −3 and 3, in particular between −2 and 2;
B1 is comprised between 1 and 5, in particular between 2 and 4;
B2 is comprised between 0 and 4.5, in particular between 0.5 and 3;
B3 is comprised between −2 and 2, in particular between −1 and 1; and
B4 is comprised between −1 and 2, in particular between 0 and 2.

3. The computer program according to claim 2, wherein the computer program causes a step before step (a) to stock the information relating to the levels of hsa-miR-34a-5p and YKL-40 measured at three or more timepoints of a subject with MASH and the information relating to the gender of the subject.

4. The computer program according to claim 2, wherein the values of features selected in the group consisting of X1, X2, X3, X4, X5, X6, X7, X8, X9, X10, X11, X16, X17, X18, X19, X21, X24, and X27 are standardized.

5. The computer program according to claim 2, wherein the score S2 is obtained by combining the values of 23 features in a logistic function:
$$S 2 = \frac{{e}^{y}}{1 + {e}^{y}}$$
Wherein
$$y = β0 + {\sum }_{n = 1}^{23} β n \times Xn$$
β0 is comprised between −0.65215 and −0.5151, in particular between −0.6339 and −0.5380;
β1 is comprised between 0.0058 and 0.0074, in particular between 0.0059 and 0.0068;
β2 is comprised between −0.1184 and −0.0907, in particular between −0.1117 and −0.1069;
β3 is comprised between −0.0565 and −0.0524, in particular between −0.0546 and −0.0527;
β4 is comprised between 0.0709 and 0.0900, in particular between 0.0815 and 0.0865;
β5 is comprised between 0.00068 and 0.00103, in particular between 0.00075 and 0.00097;
β6 is comprised between 0.0156 and 0.0182, in particular between 0.0159 and 0.0170;
β7 is comprised between −0.0799 and −0.0707, in particular between −0.0794 and −0.0715;
β8 is comprised between −0.1201 and −0.1084, in particular between −0.1156 and −0.1085;
β9 is comprised between −0.0961 and −0.0906, in particular between −0.0954 and −0.0933;
β10 is comprised between 0.0687 and 0.1002, in particular between 0.0785 and 0.0881;
β11 is comprised between 0.0153 and 0.0189, in particular between 0.0161 and 0.0180;
β12 is comprised between 0.0324 and 0.0424, in particular between 0.0375 and 0.0395;
β13 is comprised between 0.5457 and 0.6241, in particular between 0.5658 and 0.6100;
β14 is comprised between −1.1548 and −0.9475, in particular between −1.1251 and −0.9651;
β15 is comprised between −0.1768 and −0.1518, in particular between −0.1719 and −0.1631;
β16 is comprised between −0.1338 and −0.1128, in particular between −0.1331 and −0.1155;
β17 is comprised between −1.0343 and −0.7174, in particular between −0.9747 and −0.8239;
β18 is comprised between 0.0463 and 0.0539, in particular between 0.0466 and 0.0529;
β19 is comprised between −0.00129 and −0.00125, in particular between −0.00129 and −0.00127;
β20 is comprised between 0.3786 and 0.4439, in particular between 0.3993 and 0.4316;
β21 is comprised between −0.3022 and −0.2804, in particular between −0.2969 and −0.2817;
β22 is comprised between −0.4213 and −0.2989, in particular between −0.3665 and −0.3542;
β23 is comprised between 0.1744 and 0.2377, in particular between 0.1937 and 0.2235;
wherein the score S3 is obtained by combining the values of 8 features in a logistic function:
$$S 3 = \frac{{e}^{y}}{1 + {e}^{y}}$$
Wherein y=P0+P1×X2+P2×X5+P3×X24+P4×X25+P5×X26+P6×X17+P7× X27+P8×X22
Wherein:
P0 is comprised between −0.0220 and −0.0196, in particular between −0.0200 and −0.0198;
P1 is comprised between −0.1459 and −0.1154, in particular between −0.1418 and −0.1354;
P2 is comprised between −0.0475 and −0.0351, in particular between −0.0469 and −0.0427;
P3 is comprised between −0.0643 and −0.0556, in particular between −0.0627 and −0.0568;
P4 is comprised between −0.1585 and −0.1109, in particular between −0.1490 and −0.1281;
P5 is comprised between 0.3121 and 0.4023, in particular between 0.3354 and 0.3927;
P6 is comprised between −0.5694 and −0.4475, in particular between −0.5274 and −0.4683;
P7 is comprised between −0.2426 and −0.2124, in particular between −0.2404 and −0.2284;
P8 is comprised between −0.6328 and −0.4951, in particular between −0.5858 and −0.5126.

6. The computer program according to claim 5, wherein the reference S2 score is comprised between 0.4160 and 0.7238.

7. The computer program according to claim 6, wherein the reference S2 score is comprised between 0.4210 and 0.7076.

8. The computer program according to claim 5, wherein the reference S3 score is comprised between 0.5054 and 0.5954.

9. The computer program according to claim 8, wherein the reference S3 score is comprised between 0.5122 and 0.5844.

10. A computer readable medium comprising the computer program according to claim 2.

11. A system for evaluating the efficacy of a treatment of metabolic dysfunction-associated steatohepatitis (MASH) and/or for predicting the resolution of MASH and the improvement of liver fibrosis in a subject with MASH with the aid of a digital computer, for use in determining whether said subject with MASH achieves MASH resolution and/or liver fibrosis improvement, said system comprising:
a database;
at least one central processing unit; and
a memory comprising the computer program according to claim 2.
